(19) United States
(12) Patent Application Publication
(10) Pub. No.: US 2025/0375228 A1
(43) Pub. Date: Dec. 11, 2025

(54) EXPANSILE IMPLANTS FOR ORTHOPAEDIC SURGERY

(71) Applicants: Evan L. HARRIS, San Antonio, TX (US); Adam I. HARRIS, San Antonio, TX (US)

(72) Inventors: Evan L. HARRIS, San Antonio, TX (US); Adam I. HARRIS, San Antonio, TX (US)

(21) Appl. No.: 19/229,553

(22) Filed: Jun. 5, 2025

Related U.S. Application Data
(60) Provisional application No. 63/657,358, filed on Jun. 7, 2024.

(51) Int. Cl. A61B 17/74; A61B 17/00; A61B 17/72; A61F 2/30; A61F 2/36; A61F 2/38

(52) U.S. Cl. CPC A61B 17/744; A61B 17/7291; A61F 2/3662; A61F 2/3859; A61B 2017/00862; A61B 2017/00867; A61B 2017/00871; A61B 2017/00889; A61B 2017/00893; A61F 2002/3006; A61F 2002/30075; A61F 2002/30092; A61F 2002/30677

(57) ABSTRACT

In one or more embodiments orthopaedic implants may be provided. The implants may include an expansile structure which may allow for increased contact between an endosteal or periosteal surface for initial fixation and further allow for bone in-growth and/or on-growth. In some embodiments the expansile structure may be made of, for example a NiTiNol structure, which may further be programmed for expansion at body temperatures. In some embodiments the interstices of the expansile structure may further be filled with a shape memory polymer and/or other elastic material which may also be programmed for expansion at body temperatures.

### BACKGROUND

In many orthopeadic implants micromotion at the interface between the bone and the implant can lead to failure of osseous integration, gross loosening, and ultimate failure of the implant thus requiring additional revision surgeries. Some patient factors such as obesity and anatomic variation are beyond the design parameters of traditional implants. Increased contact area is associated with better initial fixation, less micromotion, and better osseous integration leading to better survivorship. However, in current implants estimates of the contact between, for example, a femoral hip stem and the proximal femur are, at best, about 60% of the available area.

Some expansile orthopaedic implants do exist, however they are limited in use to specific applications such as the treatment of tumors in the long bones of children. In addition to their limited applications these existing implants are also limited to expanding longitudinally.

### SUMMARY

In one or more embodiments expansile implants for orthopaedic surgery may be provided. The expansile implants may include an expanding structure which may allow for increased contact between an endosteal or periosteal bone surface for initial fixation and may further allow for bone in-growth and/or on-growth. In some embodiments the expanding structure may be made of, for example a NiTiNol structure, which may further be designed for expansion at body temperatures. In some embodiments the interstices of the fiber metal mesh may further be filled with a shape memory polymer and/or other elastic material which may also be programmed for expansion at body temperatures.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary implant with mesh.

FIG. 2 shows another exemplary implant.

FIG. 3 shows another exemplary implant.

FIG. 4 shows an exemplary embodiment of an expansile implant in use.

FIG. 5 shows another exemplary embodiment of an expansile implant in use.

FIG. 6 shows an exemplary method for treating a collapsed bone segment.

FIG. 7 shows an exemplary method for treating a long bone, short bone, or sesamoid fracture

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

In one or more exemplary embodiments expansile implants for orthopaedic surgery may be provided.

In an embodiment, an expansile implant may include an intra medullary stem, rod, or post. In some embodiments, an exemplary femoral stem for a hip replacement may mimic the five groups of trabeculae, which may be understood that in the native femur are formed along lines of stress, either compression or tension. The intra medullary stem, rod, or post may be mimicked by, for example, replacing each group with multiple structures of metal. The number and thickness of the structures may vary based on application. In some embodiments the intra medullary stem, rod, or post may further include a central core, which may also be made of metal. The intra medullary stem, rod, or post's metal structure may coalesce to form a trunnion upon which a traditional prosthetic femoral head might be placed. In some embodiments the intra medullary stem, rod, or post may be manufactured using 3D printing and may be made of a biological and bone compatible metal, for example titanium alloy. It may be understood that the femoral stem embodiment is for purposes of illustration and that in other embodiments the techniques and structures described may be implemented in any form of orthopaedic implant.

In some embodiments the intra medullary stem, rod, or post may further include an expansile structure, for example a metal mesh, which may allow for contact between an endosteal bone surface for initial fixation and further allow for bone in-growth and/or on-growth. In some embodiments a metal expansile structure may be made of, for example, shape memory alloy (SMA). In some embodiments the expansile structure may be configured to expand at an activation point temperature. In some embodiments the activation point temperature may be at, below, or above internal body temperature. In some embodiments the metal expansile structure may be made of, for example, a NiTiNol structure, which may further be programmed for expansion at body temperatures. In some embodiments the intra medullary stem, rod, or post may further include a non-expansile metal open structure for in-growth surrounding the expansile structure. It may be understood that the expansile structure may surround the central core. It may be understood that the expansile structure may expand in a plurality of directions including, for example, circumferentially.

In some embodiments the expansile implant may include an expansion material so as to expand to a predetermined size and shape allowing it to fit the contours of the surfaces around it. In some embodiments an interstices of the expanding structure may further be filled with a shape memory structure. In some embodiments the shape memory structure may include, for example, a shape memory polymer and/or other elastic material which may also be designed for expansion at body temperatures. In some embodiments the material may also be heated with external induction or conductive heating for activation at a point above internal body temperature so as that the shape memory structure may maintain its shape utilizing residual stiffness and activation may be achieved. It may be understood that this structure may allow for expansion and fit to the contours of an endosteal surface, or other bone surface, which may provide for increased contact area. It may further be understood that the shape memory structure may expand to a predetermined size and shape when given an appropriate input, for example heat. However, if the space available for expansion is limited by, for example, endosteal bone, the material will expand till contact, and then continue to exert a force on that bone. It may be understood that as friction is the primary force resisting micromotion, an ongoing expansile force may increase force normal to the surface and hence the resistance to motion.

In some embodiments the expansile implant may include one or more materials that dissolves over time and include a locally active compound that is distributed as the material dissolves. It may be understood that the locally active compound may be one of an antibiotic, anti-fungal, anti-tumor, anti-viral, osteoinductive or osteoconductive compound.

It may be understood that in the embodiment of the expansile implant for hip replacement, the expansile implant may include a metal structure femoral stem by decreasing the total metal amount a modulus of the implant may be reduced. By aligning the metal with the patterns of stress of a native bone applied, it may be understood that the stress would be more compressive stress rather than shear stress. As such, the fatigue tolerance may be enhanced. Furthermore, through an open design, bone may grow onto each metal trabeculum which may reinforce the trabeculae over time. The same concepts may be applied to an embodiment of any intramedullary rod for long bone fixation.

In an embodiment the femoral stem implant, or other implant, may be rendered into a standard set of implants that correspond to standard sizes and/or may be custom made for a patient based on, for example, patient specific CT data. It may be understood that a custom made implant may maximize contact between the femoral stem and the endosteal bone throughout the metaphysis and proximal diaphysis, which may distribute the stresses to a wider swath of bone. In some embodiments different holding pressure may also be utilized depending on the patient, for example increased pressures for patients with high BMI which may counteract the increased odds of micro-motion in patients with high BMI. Current custom implants may need to sacrifice some endosteal contact area to the practicality of implantation. Expansile implants would not have this design constraint.

It may be understood that the expansile structure may be applied to a plurality of other applications. For example, in some applications the property of the material filling the interstices of the memory alloy structure may vary. In an embodiment the memory structure may include an antibiotic. As the material dissolves, a significant local dose of antibiotics may minimize the possibility of bacterial growth. If the material were also a shape memory material, it may expand as much or more than the alloy allowing for further increased contact area. If the material has osteoconductive or osteoinductive properties, it may allow for significantly different implant design. Specifically, a design that in and of itself would not have adequate fatigue tolerance, when covered with integrated bone, may have long term fatigue tolerance.

In another embodiment the shape memory materials may be designed to have a functionally graded expansion profile. In some embodiments, the expansile structure may include a functionally graded profile. This might be applied to, for example, the anterior and posterior chamfers of the femoral component of a total knee replacement to provide a clamping force on the distal femur. A graded expansion may be employed here so as to accommodate imperfect femoral preparation. In some embodiments other constant materials or functional gradation may further be utilized to prevent displacement forces due to, for example, an imperfect cut to the bone.

In some embodiments, the expansile implant may be designed for fractures including one or more of tubular bone fractures, femoral neck fractures, or short bone fractures, in another embodiment in a long bone fracture, an intramedullary device may be fixed to the bone above and below the fracture with transverse screws. The quality of the bone or the potential location of the screws may sometimes be limited. Application of an expansile memory material near the ends or along the length of the implant may enhance fixation. Application of a material that also has osteoinductive properties along the shaft of the implant may decrease the rate of non-union. Antibiotics in the material may decrease the likelihood of infection in treating open fractures. This is not limited to long bones. Devices designed for femoral neck fractures or scaphoid fractures, as an example, could benefit by the same mechanisms.

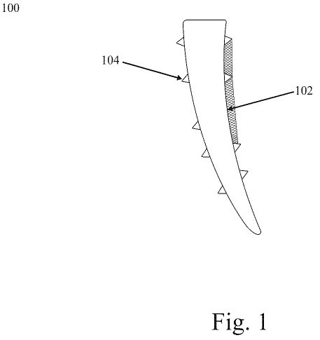

Referring to FIG. 1, an exemplary implant may be shown and described. The exemplary implant may include an implant body 102 and an expansile structure 104, which may be utilized for initial contact with the patient's bone. In some embodiments the implant body 102 may be an intra medullary stem, rod, or post.

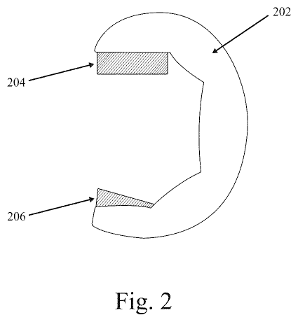

Referring to FIG. 2, another exemplary implant may be shown and described. The implant may include an implant body 202, for example a femoral component of a knee replacement, and may further include a shape memory alloy panel and/or grip surface 204. If the expansile material is of a uniform thickness and expansile property 204, clamping may be obtained. If the expansile material is graded 206, it may also provide increased resistance to displacement.

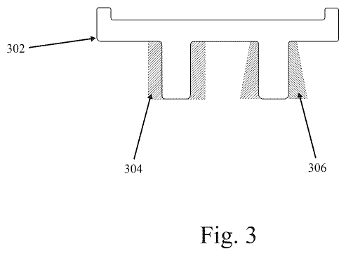

Referring to FIG. 3, another exemplary orthopaedic implant 302, for example a tibial component of a knee replacement, may be shown and described. The orthopaedic implant 302 may sit, in large part, on medullary surfaces and may have one or more pegs for fixation. In an embodiment expansile material may be added to the one or more pegs, and may increase the stability of the one or more pegs. The expansile material may be, for example, a symmetric expansile coating 304 or a graded expansile coating 306.

The concept of expansile implants may also be applied in areas other than fixation. For example they may play a role in support of a surface. Two applications may be, for example, in avascular necrosis of the hip or the shoulder, and compressed joint fractures. In the first example, the dead bone would be removed in a standard fashion, then the implant may be deployed inside the bone providing support for the compressed surface. The interstices of the implant may also be filled with a bone graft material to facilitate new bone formation. The second setting in this example would be a depressed tibial plateaux fracture. The traditional treatment is to surgically elevate the depressed surface to its anatomic position, then graft behind it. Bone grafts do not provide full support during healing so protected weight bearing is required. If the surface could be supported by an expansile implant, there would be less need for protected weight bearing.

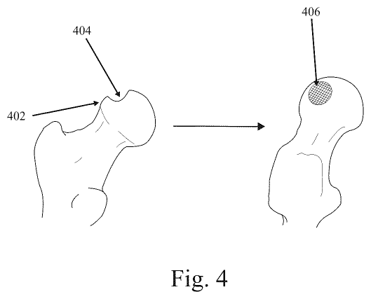

Referring to FIG. 4, an exemplary embodiment of an expansile implant being used may be shown and described. In the embodiment a femoral head 402 may have a collapsed segment 404. An expansile implant 406 may be used to repair the femoral head, such that the expansile implant 406 supports the bone. It may be understood that the femoral head is used for exemplary purposes and other collapses in other bones may be likewise treated.

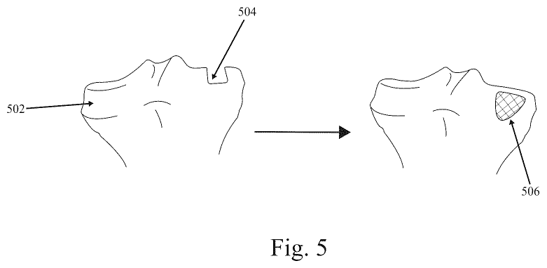

Referring to FIG. 5, another exemplary embodiment of an expansile implant being used may be shown and described. In the embodiment a tibia 502 may have a depressed area 504. An expansile implant 506 may be used to repair the tibia, such that the expansile implant 506 supports the bone. It may be understood that the tibia is used for exemplary purposes and other collapses in other bones may be likewise treated.

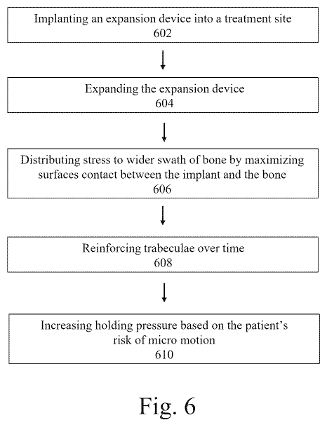

Referring to FIG. 6, a flow chart of an exemplary method for treating a collapsed bone segment may be shown. In some embodiments an expansion device may include an implant made of a biological and bone compatible metal and an expansion material that is configured to expand to a predetermined size and shape so as to support the contours of the surfaces around it. In some embodiments the implant may include an open structure and may be configured to allow bone to grow around, between, or through metal trabeculae. In some embodiments the expansion material is heated with external induction, conductive heating, or induced joule heating.

In a first step 602 the expansion device may be implanted into a treatment site. In a next step 604 the expansion device may be expanded. In a next step 606 stress to wider swath of bone may be distributed by maximizing surface contact between the implant and the bone. In a next step 608 the trabeculae may be reinforced over time. In a final step 610 holding pressure may be increased based on a patient's risk of micro motion.

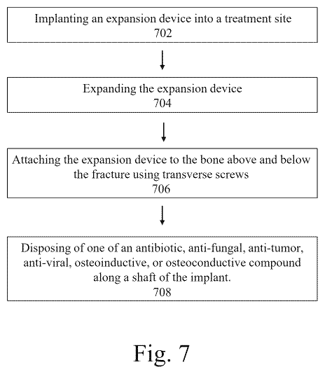

Referring to FIG. 7, a flow chart of an exemplary method for treating a long bone, short bone, or sesamoid fracture may be shown.

In a first step 702 the expansion device may be implanted into a treatment site. In a next step 704 the expansion device may be expanded. In a next step 706 the expansion device may be attached to the bone above and below the fracture using transverse screws. In a final step 708 one of an antibiotic, anti-fungal, anti-tumor, anti-viral, osteoinductive, or osteoconductive compound may be disposed along a shaft of the implant.

The design of the expansion may take many forms. An input of some sort may be required to activate the memory material. This may be, for example, the heat of body temperature, or a lower temperature where the device would be shipped in a container or shell to maintain the pre-expansile shape for implantation.

It may be understood that the above methods and embodiments may also be applied to, for example, other orthopaedic implants such as intra-medullary implants or resurfacing implants and/or veterinary applications.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An expansile implant comprising:
an intra medullary stem, rod, or post made of a biological and bone compatible metal; and
an expansion material that is configured to expand to a predetermined size and shape allowing it to fit the contours of the surfaces around it;
wherein the expansion force causes friction against one or more nearby surfaces increasing resistance to micromotion.

2. The expansile implant of claim 1, wherein the intra medullary stem, rod, or post comprises:
a central core; and
a trunnion configured to hold a prosthetic femoral head.

3. The expansile implant of claim 1, wherein the intra medullary stem, rod, or post further comprises a central core and a metal expansile structure made of shape memory alloy (SMA); and
the expansile structure is configured to expand at an activation point temperature.

4. The expansile implant of claim 2, wherein the intra medullary stem, rod, or post further comprises:
a non-expansile metal open structure for in-growth surrounding the expansile structure,
wherein the expansile structure surrounds the central core.

5. The expansile implant of claim 3, wherein the expansile structure is configured to expand in a plurality of directions including at least circumferentially.

6. The expansile implant of claim 5, wherein an interstices of the expanding structure are filled with a shape memory structure,
wherein the shape memory structure is a shape memory polymer and/or other elastic material; and
the shape memory structure is configured to expand to a predetermined size.

7. The expansile implant of claim 6, wherein the activation point temperature is at or below internal body temperature.

8. The expansile implant of claim 6, wherein the expansile implant includes one or more materials that dissolves over time and include a locally active compound that is distributed as the material dissolves.

9. The expansile implant of claim 8, wherein the locally active compound is one of an antibiotic, anti-fungal, anti-tumor, anti-viral, osteoinductive or osteoconductive compound.

10. The expansile implant of claim 6, wherein the shape memory or the expansile structure has a functionally graded profile.

11. The expansile implant of claim 6, wherein the expansile implant is a hip replacement comprising:
a metal structure femoral stem,
wherein the total amount of metal and a modulus of the implant are reduced,
wherein the metal is aligned with the stress patterns of a native bone; and
the metal modulus is adjusted based on a patient's risk of micro motion.

12. The expansile implant of claim 6, wherein the expansile implant is designed for fractures including one or more of tubular bone fractures, femoral neck fractures, or short bone fractures.

13. The expansile implant of claim 6, wherein the activation point is above internal body temperature,
wherein the shape memory structure maintains its shape using residual stiffness and activation is achieved by heating with external induction, conductive heating, or induced joule heating.

14. The expansile implant of claim 2, wherein the intra medullary stem, rod, or post is 3D printed.

15. A method for treating a collapsed bone segment comprising:
implanting an expansion device into a treatment site;
expanding the expansion device,
wherein the expansion device comprises:
an implant made of a biological and bone compatible metal; and
an expansion material that is configured to expand to a predetermined size and shape configured to support the contours of surfaces around it.

16. The method for treating a collapsed bone segment of claim 15 further comprising:
distributing stress to a wider swath of bone by maximizing surface contact between the implant and the bone.

17. The method for treating a collapsed bone segment of claim 15 further comprising:
reinforcing a trabeculae over time,
wherein the implant includes an open structure and is configured to allow bone to grow around, between, or through the metal trabeculae.

18. The method for treating a collapsed bone segment of claim 15 further comprising:
increasing holding pressure based on a patient's risk of micro motion.

19. The method for treating a collapsed bone segment of claim 15, wherein the expansion material is heated with external induction, conductive heating, or induced joule heating.

20. A method for treating a long bone, short bone, or sesamoid fracture comprising:
implanting an expansion device into a treatment site;
expanding the expansion device,
wherein the expansion device comprises:
an implant made of a biological and bone compatible metal; and
an expansion material that is configured to expand to a predetermined size and shape allowing it to maintain alignment and compression of one or more fracture fragments.

21. The method for treating the long bone, short bone, or sesamoid fracture of claim 20, further comprising:
attaching the expansion device to the bone above and below the fracture using transverse screws; and
disposing of one of an antibiotic, anti-fungal, anti-tumor, anti-viral, osteoinductive, or osteoconductive compound along a shaft of the implant.
